(19) United States
(12) Patent Application Publication
(10) Pub. No.: US 2024/0308040 A1
(43) Pub. Date: Sep. 19, 2024

(54) DISASSEMBLING DEVICE FOR A PHYSIOLOGICAL SIGNAL MONITORING DEVICE

(71) Applicant: BIONIME CORPORATION, Taichung City (TW)

(72) Inventors: Chun-Mu HUANG, Taichung City (TW); Hsueh-Chuan CHANG, Taichung City (TW); Hung-Wen CHIANG, Taichung City (TW)

(73) Assignee: BIONIME CORPORATION, Taichung City (TW)

(21) Appl. No.: 18/604,286

(22) Filed: Mar. 13, 2024

Related U.S. Application Data
(60) Provisional application No. 63/490,314, filed on Mar. 15, 2023.

(51) Int. Cl. B25B 27/14; A61B 5/00; A61B 5/145

(52) U.S. Cl. CPC B25B 27/14; A61B 5/0002; A61B 5/14532

(57) ABSTRACT

A disassembling device of a physiological parameter monitoring device comprises a base seat and a disassembling unit. The base seat has an accommodating recess, and the accommodating recess has an inner side end and an open end. The physiological parameter monitoring device is slidably disposed in the accommodating recess. The disassembling unit includes at least one abutment member connected to the base seat, and the abutment member extends from the inner side end toward the open end. When the physiological parameter monitoring device is slidably disposed in the accommodating recess of the base seat, the abutment member may be inserted between a transmitter and a bottom seat to thereby separate the transmitter and the bottom seat from each other, so as to achieve an effect of recycling and then reusing the transmitter.

### CROSS-REFERENCE TO RELATED APPLICATION

This application claims the benefit of U.S. Provisional Patent Application No. 63/490,314, filed on Mar. 15, 2023, which is incorporated by reference herein in its entirety.

### TECHNICAL FIELD

The present invention relates to an accessory of a physiological parameter monitoring device, and more particularly to a disassembling device of a physiological parameter monitoring device.

### PRIOR ART

In order to control diabetic condition or reduce its complications, blood sugar control is a very important part. Regularly measuring blood sugar and understanding the trend of variation in blood sugar concentration ensure that the blood sugar concentration is at a safe and stable level for a long term. In the past two decades, a continuous glucose monitoring (CGM) system that may be implanted in the body is developed most rapidly to monitor in real time and continuously the effect of insulin injection or drug taking, diet, and exercise on the blood sugar concentration throughout the day, thereby providing more effective help on treatment and control.

A basic architecture of a continuous blood glucose monitoring system at least comprises a sensor for measuring a physiological signal corresponding to glucose concentration in the body, an transmitter for receiving and transmitting the physiological signal, and a bottom seat for mounting the foregoing two to a skin surface. Because of a risk of sensitization may be caused by the sensor that is implanted under the skin, the sensor needs to be replaced every one to two weeks, which results in a financial burden for patients who require to monitor their blood sugar for a long term. Especially, the manufacturing cost of the transmitter is expensive as compared to the sensor and bottom seat. Additionally, the excessive use of disposable electronic components also causes environmental pollution, which fails to meet the requirement of social responsibility of company governance, i.e., the ESG sustainable management philosophy. Therefore, ideally, it would be a better development direction to detach the transmitter from the bottom seat and reuse it when the sensor reaches its threshold of use.

Since the continuous blood sugar monitoring system must be worn by users for a long time, miniaturization in its volume is an inevitable trend. In the industry, how to adjust the configuration of internal components of the transmitter, and to cooperate with a miniaturized transmitter to adjust its configuration with the bottom seat will be an important point to be developed in the future. Meanwhile, once the entire device is miniaturized, how to allow the user to easily detach the transmitter from the bottom seat for replacement is another issue.

### CONTENT OF INVENTION

Therefore, an object of the present invention is to provide a disassembling device for detaching a transmitter of a physiological parameter monitoring device and recycling and reusing the same.

Accordingly, a disassembling device of a physiological parameter monitoring device of the present invention is provided. The physiological parameter monitoring device includes a bottom seat, a sensor disposed on the bottom seat, and a transmitter separably covering the bottom seat. The bottom seat has at least one aperture. The disassembling device comprises a base seat and a disassembling unit. The base seat has an accommodating recess. The accommodating recess has an inner side end and an open end opposite to the inner side end along an axial direction. The physiological parameter monitoring device is slidably disposed in the accommodating recess along the axial direction. The disassembling unit includes at least one abutment member connected to the base seat. The abutment member extends along the axial direction and from the inner side end toward the open end. When the physiological parameter monitoring device is slidably disposed in the accommodating recess of the base seat, the abutment member passes through the aperture of the bottom seat and is inserted between the transmitter and the bottom seat, so that the transmitter and the bottom seat are separated from each other.

The effects of this invention reside in that by utilizing the disassembling device, when the sensor reaches its threshold of use, the transmitter and the sensor may be separated and detached from the bottom seat, to repeatedly use the transmitter, which is economical and may reduce the problem of environmental pollution.

### BRIEF DESCRIPTION OF THE DRAWINGS

Other features and effects of the present invention will be apparently presented in the embodying manner with reference to the drawings, in which:

FIG. 1 is an exploded perspective view of a physiological parameter monitoring device with which a disassembling accessory of the present invention is adapted to be used;

FIG. 2 is an assembled sectional view of FIG. 1;

FIG. 3 is a sectional view taken along line III-III in FIG. 2;

FIG. 4 is a schematic exploded perspective view of a first embodiment of a disassembling device of the physiological parameter monitoring device according to the present invention;

FIG. 5 is a schematic view of a planar operation action of the first embodiment;

FIG. 6 is a schematic view of a planar operation that is completed of the first embodiment;

FIG. 7 is a sectional view taken along line VII-VII in FIG. 4;

FIG. 8 is a schematic side view of the first embodiment;

FIG. 9 is a sectional view taken along line IX-IX in FIG. 8;

FIG. 10 is a schematic perspective assembled view of a second embodiment of the disassembling device of a physiological parameter monitoring device according to the present invention;

FIG. 11 is a schematic perspective view of the second embodiment;

FIG. 12 is a schematic view of a planar operation action of the second embodiment; and

FIG. 13 is a schematic view of a planar operation that is completed of the second embodiment.

### EMBODYING MANNER

Before the present invention is described in detail, it should be noted that similar elements are represented by the same reference numerals in the following description

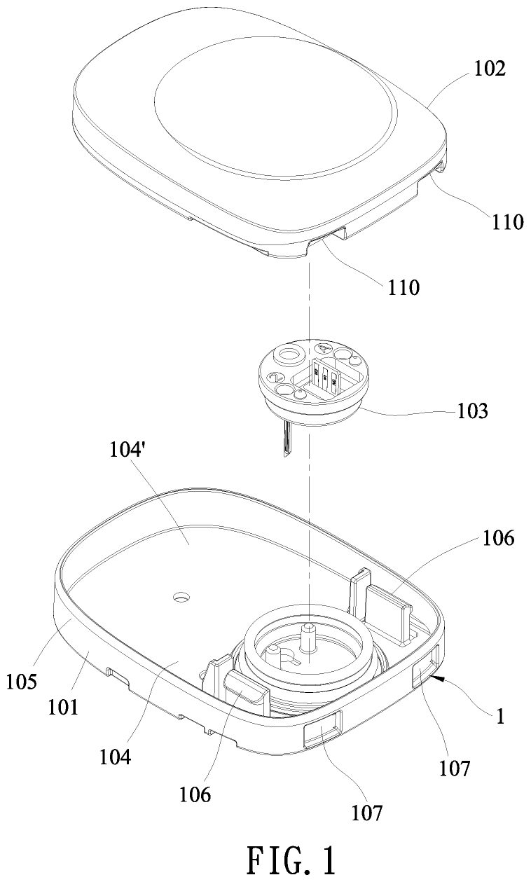

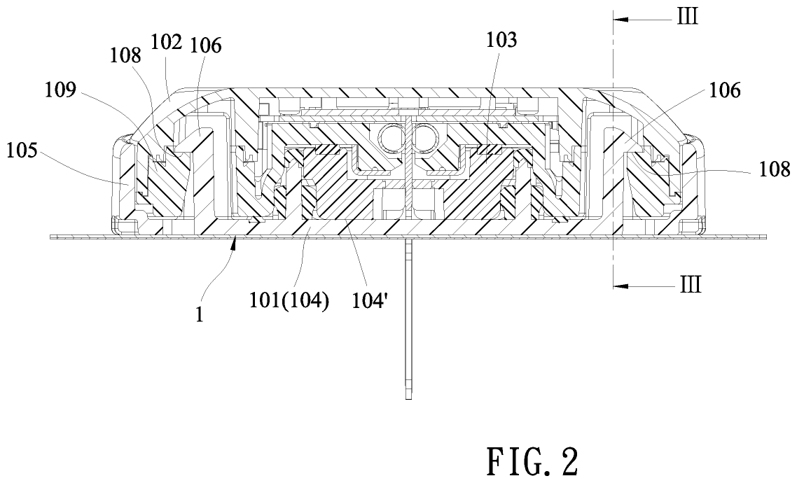

As shown in FIGS. 1 to 3, the physiological parameter monitoring device 1 includes a bottom seat 101, a sensor 103 that may be disposed on the bottom seat 101, and a transmitter 102 that separably covers the bottom seat 101. The bottom seat 101 has a bottom plate 104 to be disposed on a skin surface of an organism, a surrounding wall 105 connected to a periphery of the bottom plate 104 and extending upwardly, two first engaging structures 106 protrudingly disposed on a top surface 104′ of the bottom plate 104, and two apertures 107 respectively adjacent to the first engaging structures 106 and openly disposed in the surrounding wall 105. The sensor 103 is adapted for measuring at least one analyzed substance in the organism and transmitting a corresponding physiological signal. When covering the bottom seat 101, the transmitter 102 is electrically connected to the sensor 103 for receiving and outputting the physiological signal. In one embodiment, the bottom seat 101 and the sensor 103 may be defined as a sensor set. The sensor set is disposable as compared to the transmitter 102 that is recyclable for reuse.

Specifically, the transmitter 102 has a bottom housing 109, two second engaging structures 108 each disposed on the bottom housing 109 and engagable with the corresponding first engaging structures 106 in a mutual manner. When the transmitter 102 is covering on the bottom seat 101 with the bottom housing 109 facing the top surface 104′ of the bottom plate 104 of the bottom seat 101, the second engaging structures 108 and the first engaging structures 106 mutually engage.

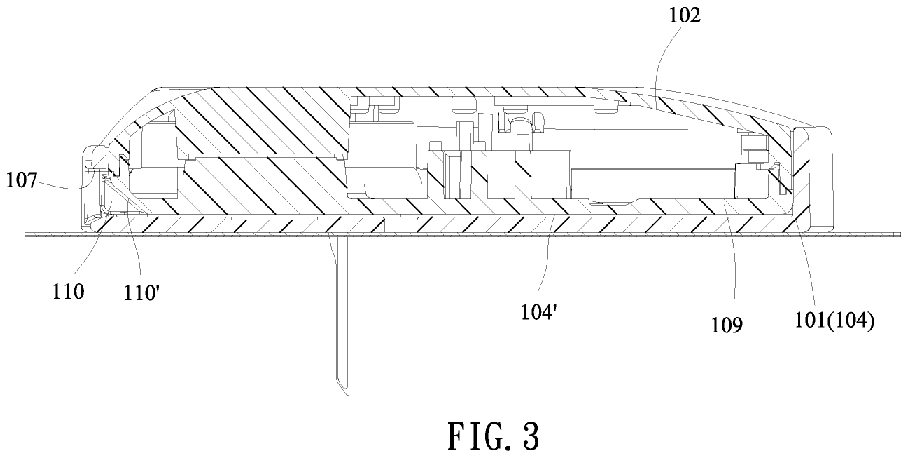

More specifically, the transmitter 102 has two notches 110 corresponding to the apertures 107. The bottom housing 109 faces the top surface 104′ of the bottom plate 104 of the bottom seat 101. In FIG. 3, a cross-sectional shape of the notches 110 is triangle, and the bottom housing 109 further has two guiding surfaces 110′ respectively adjacent to the notches 110. When the transmitter 102 is covering on the bottom seat 101 with the bottom housing 109 facing the top surface 104′ of the bottom plate 104 of the bottom seat 101, the second engaging structures 108 and the first engaging structures 106 mutually engage, and the notches 110 also face and communicate with the apertures 107, respectively.

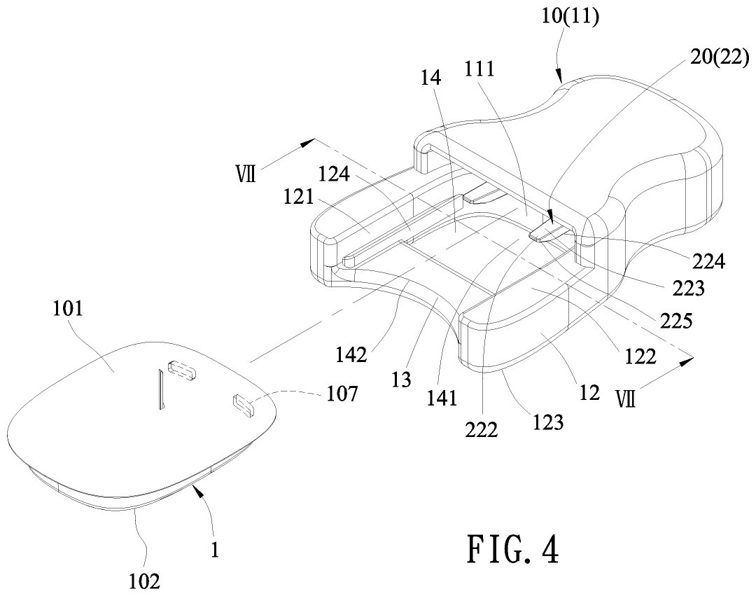

Please refer to FIGS. 4 to 6, in which it is a first embodiment of a disassembling device of a physiological parameter monitoring device 1 according to the present invention that comprises a base seat 10 and a disassembling unit 20.

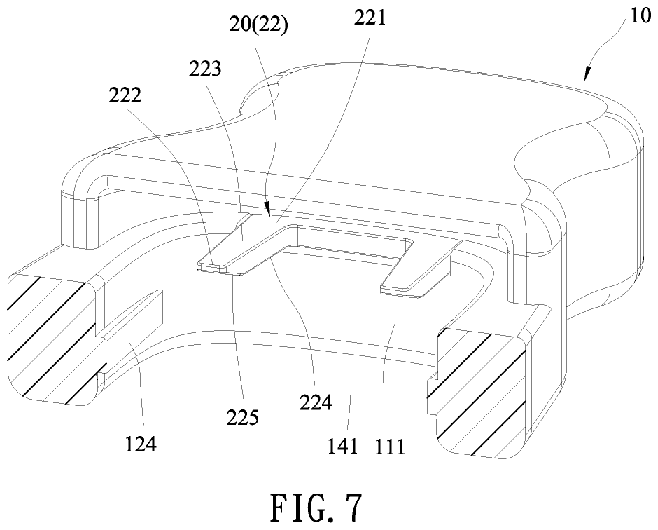

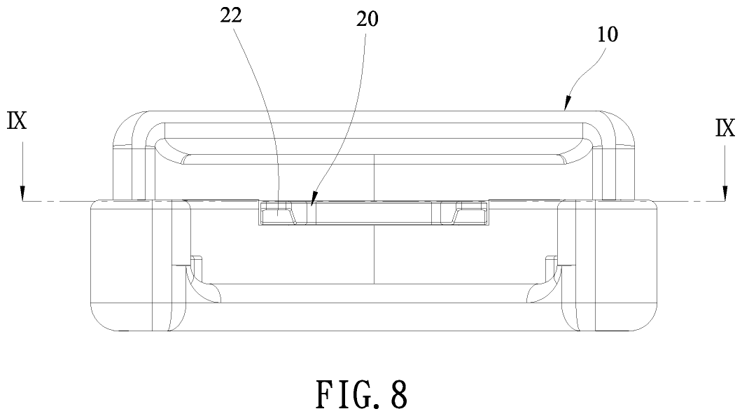

Referring to FIGS. 7 and 8, the base seat 10 includes a main body 11, a pair of extension arms 12 connected to the main body 11, and a resting plate 13 connected to the extension arms 12. The main body 11 and the extension arms 12 cooperatively define an accommodating recess 14. The accommodating recess 14 has an inner side end 141 and an open end 142 opposite to the inner side end 141 along an axial direction (X). The physiological parameter monitoring device 1 may be slidably disposed in the accommodating recess 14 along the axial direction (X). The main body 11 has an end surface 111 located at the inner side end 141 of the accommodating recess 14. Each of the extension arms 12 has an inner side surface 121 adjacent to the accommodating recess 14, a top planar surface 122 transversely connected to the inner side surface 121, a bottom planar surface 123 transversely connected to the inner side surface 121 and opposite to the top planar surface 122, and a limiting rib 124 protrudingly disposed on the inner side surface 121. The limiting ribs 124 are provided for an end edge of the surrounding wall 105 of the physiological parameter monitoring device 1 to abut thereagainst. The resting plate 13 is adjacent to the open end 142 and is adjacent to the bottom planar surface 123.

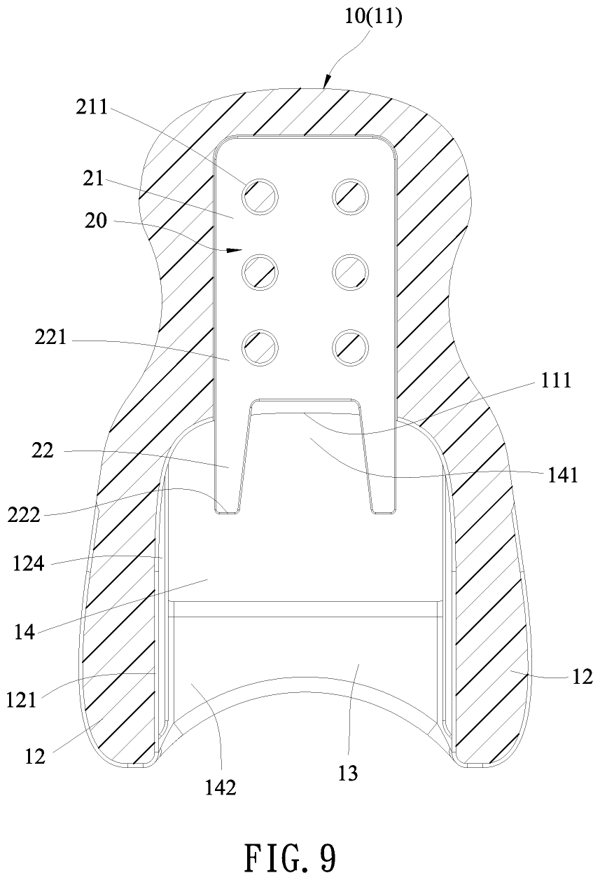

The disassembling unit 20 includes at least one abutment member 22 connected to the base seat 10. The abutment member 22 extends along the axial direction (X) and from the inner side end 141 toward the open end 142. Specifically, each of the abutment members 22 has a connecting end portion 221 connected to the base seat 10, a terminating end portion 222 opposite to the connecting end portion 221, an upper surface 223 extending from the connecting end portion 221 toward the terminating end portion 222, a lower surface 224 opposite to the upper surface 223, and a guiding inclined surface 225 connected to the lower surface 224 and corresponding to the terminating end portion 222. With reference to FIG. 9, a width of the connecting end portion 221 of each of the abutment members 22 is greater than a width of the terminating end portion 222. When the physiological parameter monitoring device is slidably disposed in the accommodating recess of the base seat, the abutment members 22 respectively pass through the apertures 107 of the base seat 1, and are inserted between the transmitter 102 and the bottom seat 101, thereby separating the transmitter 102 and the bottom seat 101 from each other.

In one embodiment, the disassembling unit 20 may further include a fixed plate member 21 embedded inside the main body 11, and an amount of the abutment members 22 is two and the abutment members 22 are connected to the base seat 10 through the fixed plate member 21. The fixed plate member 21 has a number of through holes 211, and the material for forming the base seat 10 may be filled in the through holes 211. The abutment members 22 extend along the axial direction (X) from the inner side end 141 toward the open end 142, and extend from the end surface 111 toward the open end 142.

In order to further understand the functions produced, the technical means applied, and the expected effect achieved by cooperation of components of the present invention, they will be described again below, and it is believed that a more deep and specific understanding of the present invention can be obtained thereby.

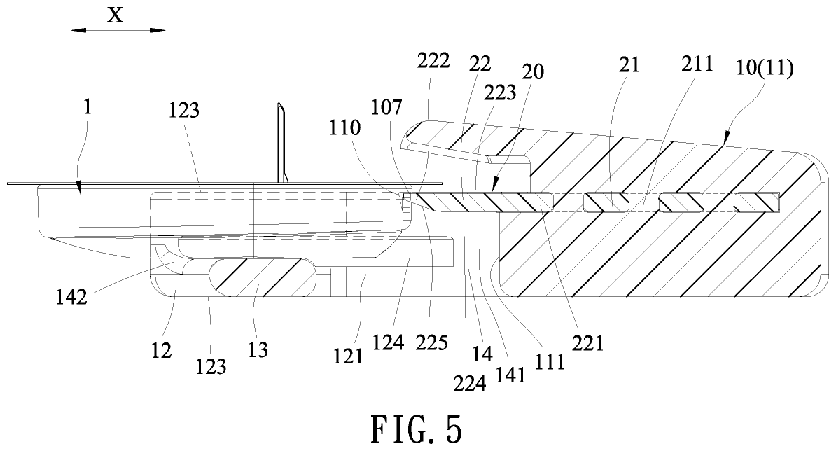

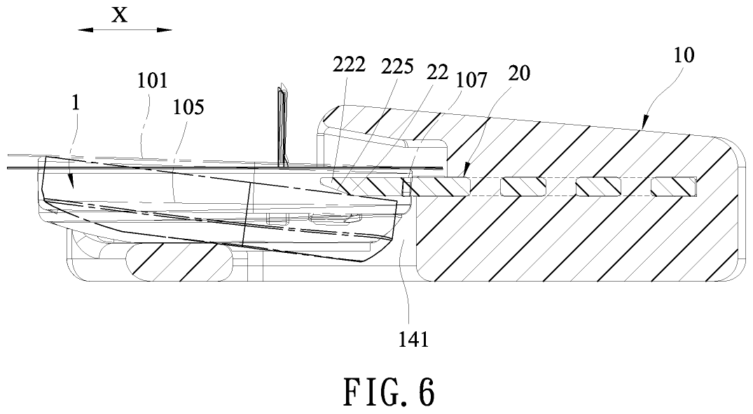

As shown in FIGS. 4 and 5, the user first orients the aperture 107 of the physiological parameter monitoring device 1 toward the inner side end 141 with the transmitter 102 facing the resting plate 13 and making the transmitter 102 abut against the resting plate 13. Referring to FIGS. 6 and 7, the user then pushes the physiological parameter monitoring device 1 along the axial direction (X) toward the inner side end 141. Since the end edge of the surrounding wall 105 of the bottom seat 101 of the physiological parameter monitoring device 1 abuts against the limiting ribs 124, the physiological parameter monitoring device 1 is guided to smoothly move toward the inner side end 141.

As shown by an imaginary line in FIG. 6, when the abutment members 22 are inserted into the apertures 107 and the notches 110 of the physiological parameter monitoring device 1, the abutment members 22 are inserted between the transmitter 102 and the bottom seat 101 to thereby separate the transmitter 102 and the bottom seat 101 from each other.

By utilizing the bottom housing 109 that has the guiding surfaces 110′ adjacent to the notches 110, each of the abutment members 22 that has a guiding inclined surface 225 corresponding to the terminating end portion 222, and the effect of the width of the connecting end portion 221 of each of the abutment members 22 being greater than the width of the terminating end portion 222 (see FIG. 9), the abutment members 22 are allowed to be smoothly inserted in the notches 110 without precisely aligning the same with the notches 110. Furthermore, the transmitter 102 may be smoothly detached from the bottom seat 101 so the transmitter 102 may be recycled.

When the sensor 103 reaches a threshold of use, the disassembling device of the present invention is utilized to easily detach the transmitter 102 from the bottom seat 101 for reuse. Additionally, since a manufacturing cost of the transmitter 102 is expensive as compared to that of the sensor 103 and bottom seat 101, in addition to reducing pollution to the environment, reuse of the transmitter 102 may also meet the requirements of the social responsibility of company governance. Moreover, the users may easily detach the transmitter 102 from the bottom seat 101 for replacement, which also reduces the financial burden of the users.

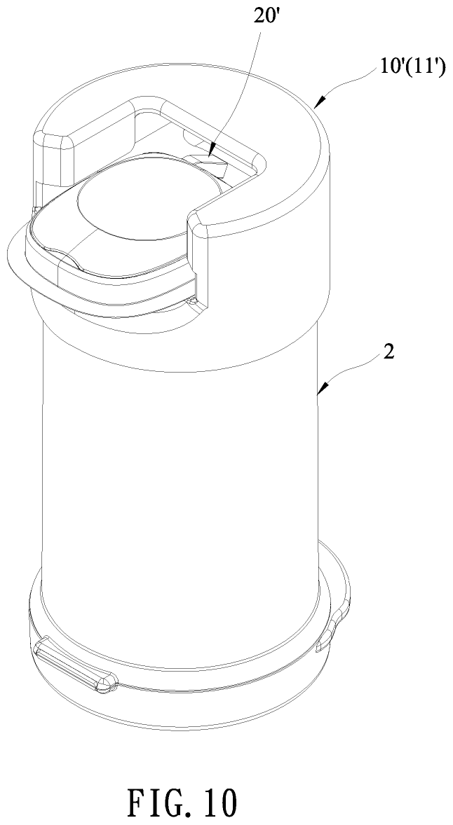

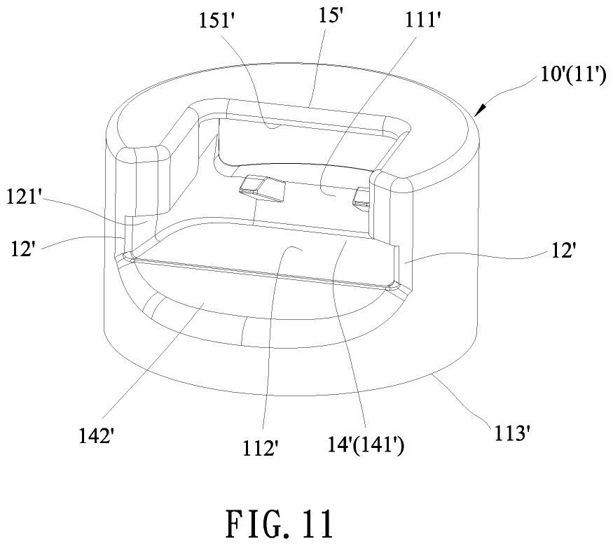

Referring back to FIGS. 10 to 13, a second embodiment of the disassembling device of the physiological parameter monitoring device is adapted to be sleeved on a drying can 2. The disassembling device also comprises a base seat 10′ and a disassembling unit 20′.

The base seat 10′ includes a main body 11′, a pair of extension arms 12′ connected to the main body 11′, and a cover plate 15′ connected to the extension arms 12′. The main body 11′, the extension arms 12′ and the cover plate 15′ cooperatively define the accommodating recess 14′. The main body 11′ has an end surface 111′ located at an inner side end 141′ of the accommodating recess 14′. Each of the extension arms 12′ has an inner side surface 121′ adjacent to the accommodating recess 14′. The cover plate 15′ has a bottom side surface 151′ adjacent to the accommodating recess 14. The main body 11′ further has a recess bottom surface 112′ adjacent to the accommodating recess 14′ and transversely connected to the end surface 111′, a bottom end surface 113′ opposite to the recess bottom surface 112′, and an inserted recess 114′ recessed in the bottom end surface 113′. The drying can 2 is inserted in the inserted recess 114′. The recess bottom surface 112′ is inclined in shape, and gradually inclines toward the bottom end surface 113′ when extending from the open end 142 toward the inner side end 141.

The abutment members 22′ are connected to the end surface 111′ of the base seat 10′, and each of the abutment members 22′ is inclinedly disposed with respect to the recess bottom surface 112′. Each of the abutment members 22′ has a connecting end portion 221′ connected to the end surface 111′, a terminating end portion 222′ opposite to the connecting end portion 221′, an upper surface 223′ extending from the connecting end portion 221′ toward the terminating end portion 222′, a lower surface 224′ opposite to the upper surface 223′, and a guiding inclined surface 225′ connected to the upper surface 223′ and corresponding to the terminating end portion 222′. Each of the abutment members 22′ is gradually away from the recess bottom surface 112′ when extending from the connecting end portion 221′ toward the terminating end portion 222′. A width of the connecting end portion 221′ of each of the abutment members 22′ is greater than a width of the terminating end portion 222′.

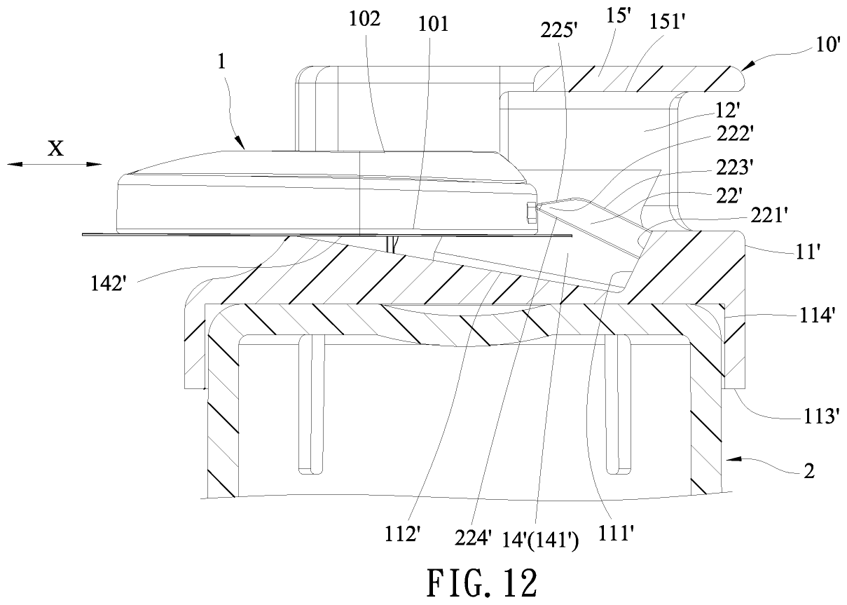

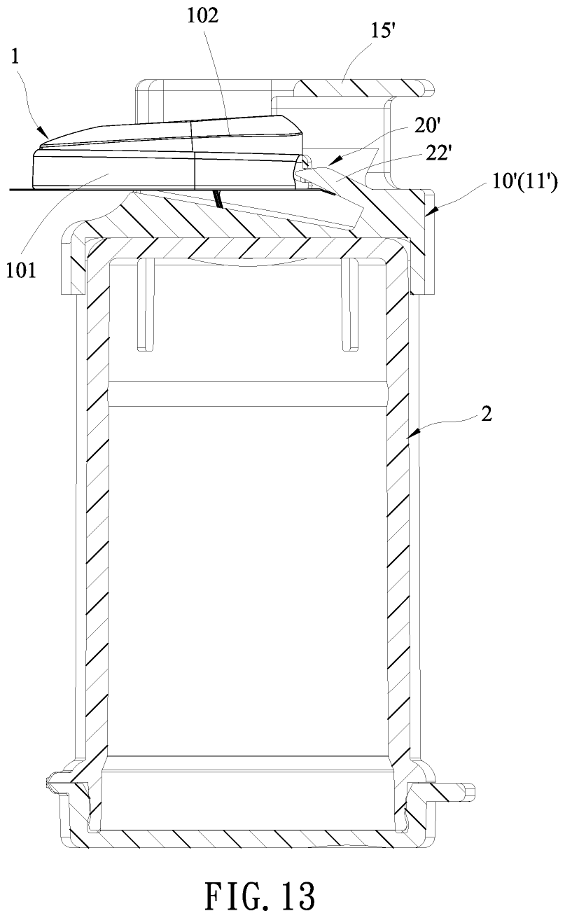

Referring to FIGS. 12 to 13, in the second embodiment, when the user encases the physiological parameter monitoring device 1 along the recess bottom surface 112′, the transmitter 102 may be smoothly detached from the bottom seat 101 by utilizing the abutment members 22′, which allows the transmitter 102 to be recycled (descriptions of smoothly detaching the transmitter 102 from the bottom seat 101 by utilizing the abutment members 22′ are described as above and will not be repeated again).

By utilizing the drying can 2 that is inserted into the inserted recess 114′, the drying can 2 and the disassembling device may be integrated together, and the drying can 2 may contain the transmitter 102 and a charger thereof (not shown) to facilitate the user to carry and storage.

In summary, the disassembling device of the physiological parameter monitoring device 1 of the present invention has a simple overall structure, allows the transmitter 102 to be reused, is cost effective, and may reduce the problem of environmental pollution, which indeed achieves the object of the present invention.

The above is merely the embodiments of the present invention, and certainly the scope of the claims of the present invention cannot be limited thereby. Any simple equivalent variation and modification made according to the claims of the present invention and the patent specification should fall within the scope covered by the claims of the present invention.

1. A disassembling device of a physiological parameter monitoring device, the physiological parameter monitoring device including a bottom seat, a sensor disposed on the bottom seat, and a transmitter separably covering the bottom seat, the bottom seat having at least one aperture, the disassembling device comprising:
a base seat that has an accommodating recess, the accommodating recess having an inner side end and an open end opposite the inner side end along an axial direction, the accommodating recess being provided for the physiological parameter monitoring device to be slidably disposed in the accommodating recess along the axial direction; and
a disassembling unit that includes at least one abutment member connected to the base seat, the abutment member extending along the axial direction and from the inner side end toward the open end;
wherein, when the physiological parameter monitoring device is slidably disposed in the accommodating recess of the base seat, the at least one abutment member passes through the at least one aperture of the bottom seat and is inserted between the transmitter and the bottom seat, to thereby separate the transmitter and the bottom seat from each other.

2. The disassembling device of the physiological parameter monitoring device as claimed in claim 1, wherein:
the bottom seat of the physiological parameter monitoring device has:
a bottom plate that is to be disposed on a skin surface of an organism;
a surrounding wall that is transversely connected to the bottom plate, the at least one aperture being disposed in the surrounding wall; and
at least one first engaging structure that is disposed on a top surface of the bottom plate; and
the transmitter has:
a bottom housing that faces the top surface of the bottom plate of the bottom seat and that has at least one notch corresponding to the at least one aperture; and
at least one second engaging structure that corresponds to the at least one first engaging structure of the bottom seat and that is disposed on the bottom housing;
wherein when the transmitter covers the bottom seat with the bottom housing facing the top surface of the bottom plate of the bottom seat, the at least one second engaging structure and the at least one first engaging structure mutually engaging, and
wherein the at least one abutment member pushes the bottom housing of the transmitter when passing through the at least one aperture of the bottom seat and being inserted into the at least one notch, which separates the at least one second engaging structure from the at least one first engaging structure, so as to allow the transmitter to be separated from the bottom seat.

3. The disassembling device of the physiological parameter monitoring device as claimed in claim 1, wherein the base seat includes a main body, and a pair of extension arms connected to the main body, the main body and the extension arms cooperatively defining the accommodating recess, the main body having an end surface that is located at an inner side end of the accommodating recess, each of the extension arms having an inner side surface that is adjacent to the accommodating recess, the at least one abutment member extending from the end surface of the base seat toward the open end.

4. The disassembling device of the physiological parameter monitoring device as claimed in claim 3, wherein the base seat further includes a resting plate connected to the extension arms.

5. The disassembling device of the physiological parameter monitoring device as claimed in claim 4, wherein each of the extension arms of the base seat further includes a top planar surface transversely connected to the inner side surface, and a bottom planar surface transversely connected to the inner side surface and opposite to the top planar surface, the resting plate being adjacent to the open end and being adjacent to the bottom planar surface.

6. The disassembling device of the physiological parameter monitoring device as claimed in claim 4, wherein each of the extension arms of the base seat further has a limiting rib protrudingly disposed on the inner side surface, the limiting ribs being provided for an end edge of the surrounding wall of the physiological parameter monitoring device to abut thereagainst.

7. The disassembling device of the physiological parameter monitoring device as claimed in claim 1, wherein an amount of the aperture of the bottom seat is two, an amount of the abutment members of the disassembling unit is also two, and each of the abutment members of the disassembling unit has a connecting end portion connected to the base seat, a terminating end portion opposite to the connecting end portion, an upper surface extending from the connecting end portion toward the terminating end portion, a lower surface opposite to the upper surface, and a guiding inclined surface connected to the lower surface and corresponding to the terminating end portion.

8. The disassembling device of the physiological parameter monitoring device as claimed in claim 7, wherein a width of the connecting end portion of each of the abutment members of the disassembling unit is greater than a width of the terminating end portion.

9. The disassembling device of the physiological parameter monitoring device as claimed in claim 1, wherein the base seat includes a main body, a pair of extension arms connected to the main body, and a cover plate connected to the extension arms, the main body, the extension arms and the cover plate cooperatively defining the accommodating recess, the main body having an end surface that is located at an inner side end of the accommodating recess, each of the extension arms having an inner side surface that is adjacent to the accommodating recess, the cover plate having a bottom side surface that is adjacent to the accommodating recess, the at least one abutment member being connected to the end surface of the base seat.

10. The disassembling device of the physiological parameter monitoring device as claimed in claim 9, wherein the main body further has a recess bottom surface adjacent to the accommodating recess and transversely connected to the end surface, and a bottom end surface opposite to the recess bottom surface, the recess bottom surface being inclined, and gradually tilting toward the bottom side surface when extending from the open end toward the inner side end.

11. The disassembling device of the physiological parameter monitoring device as claimed in claim 10, wherein the at least one abutment member of the disassembling unit is inclinedly disposed with respect to the recess bottom surface, the at least one abutment member having a connecting end portion that is connected to the end surface, and a terminating end portion that is opposite to the connecting end portion, the at least one abutment member being gradually away from the recess bottom surface when extending from the connecting end portion toward the terminating end portion.

12. The disassembling device of the physiological parameter monitoring device as claimed in claim 9, wherein the at least one abutment member of the disassembling unit has a connecting end portion connected to the end surface, a terminating end portion opposite to the connecting end portion, an upper surface extending from the connecting end portion toward the terminating end portion, a lower surface opposite to the upper surface, and a guiding inclined surface connected to the upper surface and corresponding to the terminating end portion.

13. The disassembling device of the physiological parameter monitoring device as claimed in claim 12, wherein a width of the connecting end portion of the at least one abutment member of the disassembling unit is greater than a width of the terminating end portion.

14. The disassembling device of the physiological parameter monitoring device as claimed in claim 9 adapted to be sleeved on a drying can, the main body further having a recess bottom surface that is adjacent to the accommodating recess and that is transversely connected to the end surface, a bottom end surface that is opposite to the recess bottom surface, and an inserted recess that is recessed in the bottom side surface for insertion of the dry can thereinto.
